(19) United States
(12) Patent Application Publication
(10) Pub. No.: US 2011/0046224 A1
(43) Pub. Date: Feb. 24, 2011

(54) METHODS TO ACCELERATE MUSCLE DEVELOPMENT, DECREASE FAT DEPOSITS, AND ENHANCE FEEDING EFFICIENCY IN PIGS

(75) Inventors: Ruben Herrera Herrera, Guadalajara (MX); Maria Lucia Alejo Castillo, Zapopan (MX); Ali Jesus ASAFF TORRES, Hermosillo (MX)

Correspondence Address: CONLEY ROSE, P.C.;David A. Rose, P. O. BOX 3267, HOUSTON, TX, 77253-3267, US

(73) Assignee: BIOKAB, S.A. de C.V., Mexico City (MX)

(21) Appl. No.: 12/640,248

(22) Filed: Dec. 17, 2009

(30) Foreign Application Priority Data
Aug. 20, 2009 (MX) MX/A/2009/008888

(51) Int. Cl. A61K 31/192; A61P 43/00; A23K 1/18; A23K 1/16

(52) U.S. Cl. 514/568; 426/601

(57) ABSTRACT

A natural method to promote or enhance food efficiency and muscle/fat ratio in animals is described, particularly for pigs, through the administration of an effective amount of pure ferulic acid, substantially pure ferulic acid or ferulic acid formulations. Prepared food and formulations for animals are also described, which can increase muscular development in pigs, decreasing fat deposits or promoting or increasing animal growth or enhancing food efficiency in an animal. Effective amounts of pure ferulic acid or substantially pure ferulic acid use in said food preparations and formulations are described.

### STATEMENT REGARDING FEDERALLY SPONSORED RESEARCH OR DEVELOPMENT

Not applicable

### CROSS-REFERENCE TO RELATED APPLICATIONS

This application claims benefit of Mexican patent application Serial No. MX/a/2009/008888 filed Aug. 20, 2009, which is hereby incorporated herein by reference in its entirety for all purposes.

### FIELD OF THE INVENTION

This invention refers to methods to increase the muscle/fat ratio in animals; enhance or improve growth thereof, and/or increase feeding efficiency in animals, through the therapeutic provision of food supplements formulated based on natural extracts or purified natural compounds that pertain to the group of phenolic acids or esters thereof, in amounts which are effective for this purpose. The invention also refers to compositions for using said compositions in feeding methods and food supplements for animals, which compositions include ferulic acid as active ingredient.

### PRIOR ART

Facing the enormous growth of human population and the increasing demand for food, the optimization thereof in terms of quality and quantity is crucial, through genetic enhancements and technological and pharmacological development, as the case may be.

Animal breeding has used reproductive, feeding, environmental, and pharmacologic technologies in order to achieve a higher production in shorter times. Thus, the most common practice since the 50's is to apply substances such as antibiotics, probiotics, enzymes, immunologic system stimulators, metabolic modifiers, or anabolic agents, etc. Using the latter is a widely spread practice, both within the country and abroad, in order to enhance carcass efficiency in several domestic species. β-Agonists, such as, without limitation, clenbuterol, zilpaterol, and ractopamine, from the group of phenetanolamines, are the most common. These drugs cause the activation of adrenergic β-receptors, increasing the intracellular concentrations of cyclic adenosine monophosphate (cAMP), which triggers a series of events in several cells and organs. Cell responses to β-receptor activation includes, for instance, lipolytic activity within adipose tissues, relaxing activity of the smooth muscle within the bronchi and increased heart contractions. (Goodman-Gilman, The Pharmacological basis of Therapeutics, 9th Ed, 1996, McGraw-Hill ISBN 0-07-026266-7).

Due to these features, adrenergic β-receptor agonists have therapeutic properties as bronchodilators, and also have anabolic properties since said agonists have the ability to decrease the protein reduction rate within the muscle cell, thereby causing a larger development of said muscle cell. This anabolic activity is added to the light effect this agent has on body temperature, which increases after using the compound. This increased body temperature facilitates fat consumption within the organism, causing thereby a larger efficiency and lean tissue ratio in the carcass.

Adrenergic β-receptor agonists have pharmacological and toxicological side effects, which are found within a range from lower to higher importance. For example, relaxation of bronchus smooth muscle may be considered a minor side effect in farm animals. However, the racemic mixture of ractopamine has been identified as a cause of stress in farm animals (marchand-Forde J. N., et al., The effects of ractopamine on the behaviour and physiology of finishing pigs, J. Anim. Sci., 2003, 81: 416-422). The increased level of stress in animals is crucial, since said increased level of stress increases mortality of animals during transportation. Furthermore, farm animal stress, particularly in pigs, is believed to cause the PSE syndrome (low meat quality, pale, tender and exudative meat which becomes dry when cooked).

In many animals, including farm animals, poultry and fish, stress is manifested directly or indirectly in several ways, ranging from irritability to aggression. Stress may cause negative cardiovascular side effects ranging from mild to severe, such as rapid heart rate or irregular heartbeat, which may result in sudden death. Prevalence of mortality induced by stress varies from one species to another, with some species being highly susceptible (Odeh F. M., Cadd G. G., Satterlee D. G. Genetic characterization of stress responsiveness in Japanese quail. POUM Sci., 2003, 82:31-35).

Another adrenergic β-receptor agonist is clenbuterol, so it has anabolic properties, which have encouraged the use thereof in veterinary; however, its overuse is prohibited in animals for meat consumption and may have severe consequences for the consumers thereof. Consumption of animal meat from animals treated with high amounts of clenbuterol may result in severe effects in the consumer, such as thyroid gland alterations, metabolic malfunctions or temperature intolerance. Excessive levels of this drug may further cause irregular heartbeats, nervousness, shaking, involuntary finger and/or toe trembles, headache, increased sweating, insomnia, potential muscular spasms, increased blood pressure and nausea. Due to these problems, most of the regulatory agencies in different countries have forbidden its use in animal nutrition.

Due to these and other problems resulting from the arbitrary use of drugs, a strong tendency exists to consume organic food, i.e., food produced with compounds or extracts which are 100% natural. Regarding growth stimulants, certain natural substances with chemical structures similar to adrenergic β-receptor agonists may also perform this role. Among them, certain cinnamic acid derivates, such as ferulic acid, stand out.

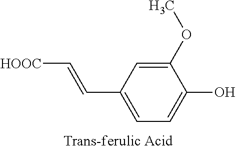

These phenolic acids and the esters thereof widely occur in nature by forming a part of the cell wall in many vegetable species, such as corn, rice, wheat, barley, oat, sugar cane, sugar beet, coffee, tomato, vegetables, citric fruits, etc.

Three methods may be used to extract ferulic acid from its natural sources: 1) low molecular weight ferulic acid compounds; 2) cell wall of plant cells; 3) tissue cultures or microbial fermentations. Ferulic acid may also be chemically synthesized from vanilla condensation and malonic acid, producing thereby a mixture of cis and trans isomers. In the first two cases, hydrolysis methods are used, whether alkaline or enzymatic. From the hydrolysis products obtained, recovery and purification processes are then performed (Fazzary y Ju; Acta Biochimica et Biophysica Sinica 2007, 39: 811-828).

A widely spread point of view is that esters and phenolic acids are considered anti-nutritional products in animal nutrition. Removal thereof or decrease of vegetable material by enzymatic means or transgenic modifications has been studied (WO 2005/096836). However, it has been remarked that, e.g., during silage, significant amounts of phenolic acids are released, thus releasing as well the digestible chains of carbohydrates, with the presence of acids not constituting a problem in animal nutrition. On the contrary, ensilaged food has important nutritional advantages over non-ensilaged food. On the other hand, little emphasis has been made on utilizing o supplementing natural phenolic compounds in animal nutrition in such a way that the use thereof constitutes an advantage for animals. The use of adrenergic β-receptor agonists is soundly controlled due to the risks said agonists represent for consumers if said agonists are used arbitrarily. The use of alternate methodologies such as the use of natural phenolic compounds or extracts containing them may be highly beneficial for this field.

### SUMMARY OF THE INVENTION

It was surprising to find that parameters such as, e.g., the amount of adipose tissue (fat) in an animal, the amount of muscular tissue (lean meat) in an animal, the growth of an animal, nutritional efficiency in an animal and muscle against fat ratio in animals, may be substantially enhanced through the administration of pure ferulic acid, substantially pure ferulic acid, ferulic acid in extracts or ferulic acid concentrates, without the risk and legal restrictions for synthetic adrenergic β-receptor agonists, since this compound is a harmless natural compound which has no adverse side effects because is part of the plant material used in animal nutrition, although it is not found in a free state. Hence, we found that the administration of effective amounts of pure ferulic acid, substantially pure ferulic acid or ferulic acid in extracts or concentrates, has the same o larger beneficial effects as growth promoter in fish breeding field, as the synthetic adrenergic β-receptor agonists such as, but not limited to, ractopamine.

Considering the foregoing, the invention provides a method promoting or enhancing animal growth in an animal, through the administration of an effective amount of pure ferulic acid, substantially pure ferulic acid or ferulic acid in extracts or concentrates.

Particularly, this invention provides a method promoting muscular growth, decreasing fat deposits or enhancing nutritional efficiency in animals, wherein said method comprises providing said animals with an effective pure ferulic acid, substantially pure ferulic acid or ferulic acid in extracts or concentrates.

In the preceding example, the invention provides a method promoting muscular growth in an animal, through the administration of an effective amount of pure ferulic acid, substantially pure ferulic acid or ferulic acid in extracts or concentrates.

In other aspect of the preceding example, the invention provides a method promoting nutritional efficiency in an animal, through the administration of an effective amount of pure ferulic acid, substantially pure ferulic acid or ferulic acid in extracts or concentrates.

In other aspect of the preceding example, the invention provides a method enhancing muscle-nutritional fat ratio in an animal, through the administration of an effective amount of pure ferulic acid, substantially pure ferulic acid or ferulic acid in extracts or concentrates.

In a further aspect of the preceding example, the invention provides compositions or formulations, which may be used in the methods, described above, which compositions or formulations include effective amounts of pure ferulic acid, substantially pure ferulic acid or ferulic acid in extracts or concentrates.

The use of this invention may also facilitate the handling of animals, particularly pigs, since it has been proven that animals treated with ractopamine racemic mixtures have frequently shown stress symptoms, thus increasing the difficulty of handling any such animals. Ferulic acid, being a harmless natural compound, has not been reported to cause a response to stress.

According to this invention, a number of advantages may be obtained from the administration in animals of a natural compound, such as pure ferulic acid, substantially pure ferulic acid or ferulic acid in extracts or concentrates, with a therapeutic activity similar to the activity of drugs such as ractopamine, zilpaterol or clenbuterol, which happen to be strictly regulated by laws and food regulatory agencies. For ensilaged food, ferulic acid occurs freely and is included in the diet of ruminant animals, with no adverse reaction reported to date. Hence, this pure compound, substantially pure compound or compound in extracts or concentrates, in compositions or formulations, may be considered a nutraceutic compound instead of a drug, and thus, this compound may be excepted from any regulations applicable in this field.

### DETAILED DESCRIPTION OF THE INVENTION

Those skilled in the art shall notice that variations and other modifications additional to those described herein may be performed on the invention. The invention includes any such variation and modification. Furthermore, the invention includes all the steps, information, formulations and compounds.

This invention comprises a method comprising the administration to animals, particularly pigs, of an effective amount of pure ferulic acid, substantially pure ferulic acid, or ferulic acid in extracts or concentrates, thus decreasing fat deposits, increasing muscular mass. and providing nutrition efficiency. Additionally, a growth enhancement is achieved (expressed as gain of weight); those skilled in the art shall notice that this growth enhancement occurs when increased muscular weight surpasses the loss of weight caused by the loss of fat tissue.

The term “effective amount” refers to the amount of compound, which is enough to obtain an intended beneficial effect. In this context, a sufficient beneficial effect is considered to be present if one or more effects described above are achieved. Particularly, a beneficial effect is considered to be present if the treatment offers a financial return at least similar to the cost of the treatment, preferably a financial return that is three times the cost of the treatment. For those skilled in the art, the effective amount shall depend on the animal species, duration of treatment and other factors.

The term “pure ferulic acid” refers to the trans-ferulic acid or any salts thereof, with a purity>98% and obtained from natural sources or chemical synthesis. The trans-ferulic acid is also known as trans-4-hydroxy-3-methoxy cinnamic acid; it is a crystalline solid with a melting point of 170° C. (Beilstein's Index: 10.436; Merck Index(14): 4062). This compound, derived from cinnamic acid, is found widely occurring in nature, forming part of cell walls in many vegetable species with no adverse side effects reported when consumed by animals or humans. On the contrary, several studies have shown that ferulic acid has several beneficial properties on both human and animal health, and is considered a nutraceutic (Fazzary and Ju; Acta Biochimica et Biophysica Sinica 2007, 39: 811-828). Apart from the multiple properties described to this point, the invention provides evidence that the ferulic acid may act as growth promoter in animals, particularly in pigs. Ferulic acid, having an elevated homology in its hydrocarbon chain with drugs such as ractopamine and clenbuterol, may act as an adrenergic β-receptor agonist; however, unlike synthetic drugs, this is a natural product having no side effects.

The term “substantially pure ferulic acid” refers to the trans-ferulic acid or any salts thereof, with >80% purity (in weight), obtained from natural sources through any obtaining or preparation methods described to date in scientific and/or patent literature. The remaining 20% generally comprises other phenolic compounds, such as p-coumaric acid, protocathecoic acid and lignocellulose residues.

The term “concentrate or extract” refers to a mean resulting from enzymatic or alkaline hydrolysis of plant materials, which mean contains released trans-ferulic acid and which mean has been treated in order to remove part of the liquefied medium where this compound is dissolved through any single method known, such as any kind of evaporation, reverse osmosis, etc.; or which as been extracted from hydrolyzed plant material using a solvent such as alcohol or ethyl acetate by any extraction method known.

This invention comprises food compositions including a mixture of food materials with pure ferulic acid or substantially pure ferulic acid, or formulations thereof in an appropriate excipient. Ferulic acid is administered preferably to animals receiving a protein-rich diet with the purpose of enhancing muscular development in those animals.

Accordingly, a part of the invention provides a nutritional preparation with high protein content supplemented with pure ferulic acid or substantially pure ferulic acid or formulations thereof in an appropriate excipient. The amount of pure ferulic acid or substantially pure ferulic acid added to the food preparation shall generally be enough to achieve concentrations between 1 and 50 ppm, preferably between 10 and 30 ppm of ferulic acid within said food preparation. The term “ppm” refers to “grams per ton”, e.g., 10 ppm are equivalent to 10 g per ton. In general, the daily intake per animal varies from 3 to 150 mg of ferulic acid, preferably from 30 to 90 mg of ferulic acid, for an average food intake of 3 kg/animal/day, generally administered during fattening or slaughter. Considering a total treatment period of 28 days, the total intake of ferulic acid per animal is 0.08 g to 4.20 g, preferably 0.84 g to 2.52 g.

When the purpose is to supplement animal nutrition with a pure ferulic acid or substantially pure ferulic acid formulation, said food supplement usually contains pure ferulic acid or substantially pure ferulic acid and an appropriate excipient or vehicle. Said supplement may be prepared by initially mixing pure ferulic acid or substantially pure ferulic acid with an appropriate excipient or vehicle. The appropriate vehicles to make the food supplement may include the following: alfalfa scone, soy scone, cotton scone, linseed scone, sodium chloride, cane molasses, urea, corn flour, nixtamalized corn flour, bone powder, rice flour, dry yeast, carboxymethyl cellulose, silicon dioxide, etc. Vehicles promote a balanced distribution of the active ingredient throughout the final food with which said vehicles are mixed. Therefore, vehicles have a very important role in order to ensure that an appropriate distribution of the active ingredient takes place throughout the food.

Pig food supplemented with ferulic acid, in the preferred embodiment thereof, generally contains from 1 to 50 g of the active ingredient per ton of food, preferably from 10 to 30 g per ton.

If the purpose is to provide animals with drinking water supplemented with ferulic acid, an appropriate vehicle may be selected which contains a determined amount of said compound in a volume of fluid, such as water, ethanol or a water-ethanol mix, which may then be added to drinking water for animals.

Drinking water for pigs supplemented with ferulic acid, in the preferred embodiment thereof, generally contains from 1 to 50 ppm of said compound.

In the example of the invention, pure ferulic acid or substantially pure ferulic acid or formulations containing ferulic acid may be administered along with one or more drugs, such as ractopamine, zilpaterol or a variety of antibiotics, with the purpose of enhancing its therapeutic activity. Said compounds or other adrenergic β-receptor agonists may be combined with the active ingredient of the invention in order to improve animal health or improve the growth-promoting activity of the formulations.

Antibiotic agents may be used along with ferulic acid in order to prevent or control infections due to bacteria, virus, fungi or other parasites. On the other hand, a plurality of antibiotics have been shown to have growth-promoting properties, although their mechanisms of action still remain unknown. Hence, antibiotic compounds may increase, promote or enhance the effects that pure ferulic acid or substantially pure ferulic acid or formulations thereof, have on reducing fat, muscular development and/or food efficiency.

The use of both growth-promoting drugs and antibiotics is restricted and limited to minimum necessary doses due to the side effects they have on animal health. Hence, the use of pure ferulic acid or substantially pure ferulic acid or formulations thereof combined with growth-promoting drugs and/or antibiotics allows for lower doses of the latter, thus decreasing the risks associated therewith.

The following studies were initiated by us and developed in animal laboratories under our close supervision:

### Example 1

### Materials and Methods

30 pigs were used, 15 castrated males and 15 females obtained from a terminal breeding York-Landrace (YL)×Duroc (DC); at the beginning of the study, said pigs weighted 77.83±1.20 Kg and were approximately 18 weeks old. Pigs were kept in individual pens, and were randomly distributed to 3 treatments, with ten repetitions per treatment. Treatments were: a positive control, including 5 ppm of commercial ractopamine; a second treatment including 12 ppm of pure ferulic acid and a third treatment containing 15 ppm of substantially pure ferulic acid per ton of food. Pigs were weighted every seven days, studying the following variables: initial weight, weekly weight, and final weight of the pigs; daily food intake. This was used to estimate the daily, weekly and term weight gain, as well as food efficiency. When reaching the approximate weight of 100 kg, animals were evaluated in vivo (using Piglot 105, SFK-Technology, Netherlands) in order to estimate the amount of body fat and lean efficiency. Results were evaluated randomly, using SAS general linear models procedures.

Results are shown in Tables 1 and 2. A similar effect can be noted in the three treatments applied throughout all the study variables: weight gain, daily gain, and food conversion. Also, a lower intake of total food and daily average in pigs supplemented with ferulic acid and ractopamine was noted when compared to the control group (which remained untreated). Furthermore, pigs fed with 15 ppm of ferulic acid showed a lower amount of dorsal fat, which resulted in a better lean efficiency (Table 2).

It is then concluded that pure ferulic acid or substantially pure ferulic acid or formulations thereof have a similar effect to customary therapeutic doses of growth-promoting drugs of the type of adrenergic β-receptor agonists, such as ractopamine.

TABLE NO. 1
Production characteristics in pigs for meat supply fed with two levels of
incorporation of ferulic acid (Mean and Standard Deviation)

| | Ractopamine | Pure ferulic acid | Substantially pure |
|---|---|---|---|
| VARIABLE | 5 ppm | 12 ppm | ferulic acid 15 ppm |
| INITIAL WEIGHT (Kg) | 77.33 ± 3.6 | 79.4 ± 5.60 | 78.2 ± 3.08 |
| FINAL WEIGHT (Kg) | 105.28 ± 4.98 | 103.85 ± 7.63 | 107.47 ± 6.84 |
| WEIGHT GAIN (Kg) | 27.94 ± 3.20 | 24.45 ± 5.64 | 29.25 ± 5.70 |
| DAILY GAIN (Kg) | 1.00 ± .08 | 0.873 ± .20 | 1.04 ± .20 |
| TOTAL INTAKE (Kg) | 82.33 ± 11.31 | 81.75 ± 14.00 | 80.57 ± 8.13 |
| AVERAGE INTAKE (Kg) | 2.35 ± .33 | 2.34 ± .40 | 2.30 ± .26 |
| CONVERSION | 2.32 | 2.68 | 2.21 |

TABLE NO. 2
Carcass quality comparison in pigs for meat supply fed with two
levels of incorporation of ferulic acid (Mean and Standard Deviation)

| | | | Substantially |
|---|---|---|---|
| | | Pure | pure |
| | Ractopamine | ferulic acid | ferulic acid |
| VARIABLE | 5 ppm | 12 ppm | 15 ppm |
| Average fat (mm) | 9.67 ± 1.11 | 13.5 ± 3.58 | 9.60 ± .34 |
| LEAN EFFICIENCY (%) | 58.98 ± 2.20 | 56.24 ± 3.58 | 59.66 ± 3.14 |
| CARCASS EFFICIENCY (%) | 87.08 | 88.01 | 89.26 |
| EFFICIENCY IN CUTS (%) | 51.69 | 49.66 | 50.88 |

1. A method promoting muscular development, decreasing fat deposits or enhancing animal food efficiency, wherein said method comprises the administration of an effective amount of ferulic acid or any salts thereof.

2. A method promoting muscular development, decreasing fat deposits or enhancing animal food efficiency, wherein said method comprises the administration of an effective amount of a natural alternative, such as ferulic acid or any salts thereof, allowing to decrease or eliminate the use of drugs usually used for these purposes.

3. The method of claim 1, wherein ferulic acid or any salts thereof is administered to animals as a food additive or supplement containing pure ferulic acid or substantially pure ferulic acid or ferulic acid extracts or concentrates in concentrations ranging from 1 to 50 ppm.

4. The method of claim 1, wherein ferulic acid or any salts thereof is administered to animals as drinking water containing pure ferulic acid or substantially pure ferulic acid or ferulic acid extracts or concentrates in concentrations ranging from 1 to 50 ppm.

5. The method of claim 1, wherein ferulic acid or any salts thereof are used to promote muscular development in farm animals.

6. The method of claim 1, wherein ferulic acid or any salts thereof are used to decrease fat deposits in farm animals.

7. The method of claim 1, wherein ferulic acid or any salts thereof are used to increase food efficiency in farm animals.

8. The method of claim 1, wherein said animals are monogastric.

9. The method of claim 1, wherein said animals are pigs.

10. The method of claim 1, wherein ferulic acid is pure and contains at least 98% in weight of trans-ferulic acid or any salts thereof.

11. The method of claim 1, wherein ferulic acid is substantially pure and contains at least 80% in weight of trans-ferulic acid or any salts thereof, and a 20% comprising coumaric acid and/or acid protocathecoic acid and/or lignocellulose residues at any ratio.

12. The method of claim 1, wherein ferulic acid is administered in the shape of plant material concentrates or extracts and/or enzymatic hydrolysis products of plant material.

13. The method of claim 1, wherein pure ferulic acid or substantially pure ferulic acid or ferulic acid extracts or concentrates are administered in combination with an effective amount of at least one antibiotic or a drug used as growth promoter such as ractopamine.

14. A food composition for animals containing a mixture of protein material, carbohydrates, fat, fiber, other nutrients and pure ferulic acid, substantially pure ferulic acid or extracts, concentrates or formulations of ferulic acid or any salts thereof.
